(19) United States
(12) Patent Application Publication
(10) Pub. No.: US 2019/0290179 A1
(43) Pub. Date: Sep. 26, 2019

(54) CONTROL APPARATUS

(71) Applicant: HONDA MOTOR CO., LTD., Tokyo (JP)

(72) Inventors: Toshiyuki MIZUNO, Wako-shi (JP); Takayuki KISHI, Wako-shi (JP); Yoshiaki KONISHI, Wako-shi (JP); Makoto KURIHARA, Wako-shi (JP)

(21) Appl. No.: 16/284,432

(22) Filed: Feb. 25, 2019

(30) Foreign Application Priority Data
Mar. 20, 2018 (JP) 2018-053099

(51) Int. Cl. A61B 5/18; B60W 50/08; B60W 30/18; H02P 29/40; B60R 11/04; B60R 21/01

(52) U.S. Cl. CPC A61B 5/18; B60W 50/08; B60W 30/18; B60R 2021/003; B60R 11/04; B60R 21/01; H02P 29/40

(57) ABSTRACT

A control apparatus for controlling a vehicle, comprising an evaluation unit configured to evaluate a degree of interest of a passenger of the vehicle with respect to time, and a setting changing unit configured to change setting of a kinetic characteristic of the vehicle based on an evaluation result obtained by the evaluation unit.

### CROSS-REFERENCE TO RELATED APPLICATION(S)

This application claims the benefit of Japanese Patent Application No. 2018-053099, filed on Mar. 20, 2018, the entire disclosure of which is incorporated herein by reference.

### BACKGROUND OF THE INVENTION

### Field of the Invention

The present invention relates to an onboard control apparatus.

### Description of the Related Art

Japanese Patent Laid-Open No. 2017-49629 describes a technique which compares and considers the contents of conflict driving operations, that is, a driving operation which brings about a feeling of pleasure and a driving operation which gives a sense of security, based on the sentiment of a passenger himself or herself, and performs driving assist corresponding to the result. According to Japanese Patent Laid-Open No. 2017-49629, it is possible to perform driving assist which does not unnecessarily hurt passenger's sentiment.

On the other hand, various situations can occur when using vehicles. As an example, it is possible that a passenger is giving priority to arriving at a destination early/he or she is in a hurry, from feelings such as pleasure described above. In a situation like this, it is necessary to consider how to implement traveling control appropriate for the passenger.

### SUMMARY OF THE INVENTION

The present invention can implement traveling control appropriate for a passenger.

One of the aspects of the present invention provides a control apparatus for controlling a vehicle, comprising an evaluation unit configured to evaluate a degree of interest of a passenger of the vehicle with respect to time, and a setting changing unit configured to change setting of a kinetic characteristic of the vehicle based on an evaluation result obtained by the evaluation unit.

Further features of the present invention will become apparent from the following description of exemplary embodiments with reference to the attached drawings.

### BRIEF DESCRIPTION OF THE DRAWINGS

FIG. 1 is a view for explaining a configuration example of a vehicle;

FIG. 2 is a block diagram for explaining a configuration example of the vehicle;

FIG. 3 is a block diagram for explaining a configuration example of the vehicle;

FIG. 4 is a flowchart for explaining an example of a method of setting the kinetic characteristic of the vehicle;

FIG. 5 is a flowchart for explaining an example of a method of evaluating the degree of interest of a passenger with respect to the time;

FIG. 6A is a schematic view for explaining an installation form example of a clock and a camera;

FIG. 6B is a schematic view for explaining an installation form example of a clock and a camera;

FIG. 6C is a schematic view for explaining an installation form example of the clock and the camera;

FIG. 7 is a timing chart for explaining an example of the method of setting the kinetic characteristic of the vehicle;

FIG. 8 is a timing chart for explaining an example of the method of setting the kinetic characteristic of the vehicle;

FIG. 9A is a timing chart for explaining an example of the method of setting the kinetic characteristic of the vehicle;

FIG. 9B is a timing chart for explaining an example of the method of setting the kinetic characteristic of the vehicle; and

### DESCRIPTION OF THE EMBODIMENTS

Embodiments of the present invention will be explained below with reference to the accompanying drawings. Note that these drawings are merely schematic views for explaining the embodiments, so the dimensions of elements in the drawings do not necessarily reflect the real dimensions. Note also that the same reference numerals denote the same elements in the drawings, and an explanation of repetitive contents will be omitted in this specification.

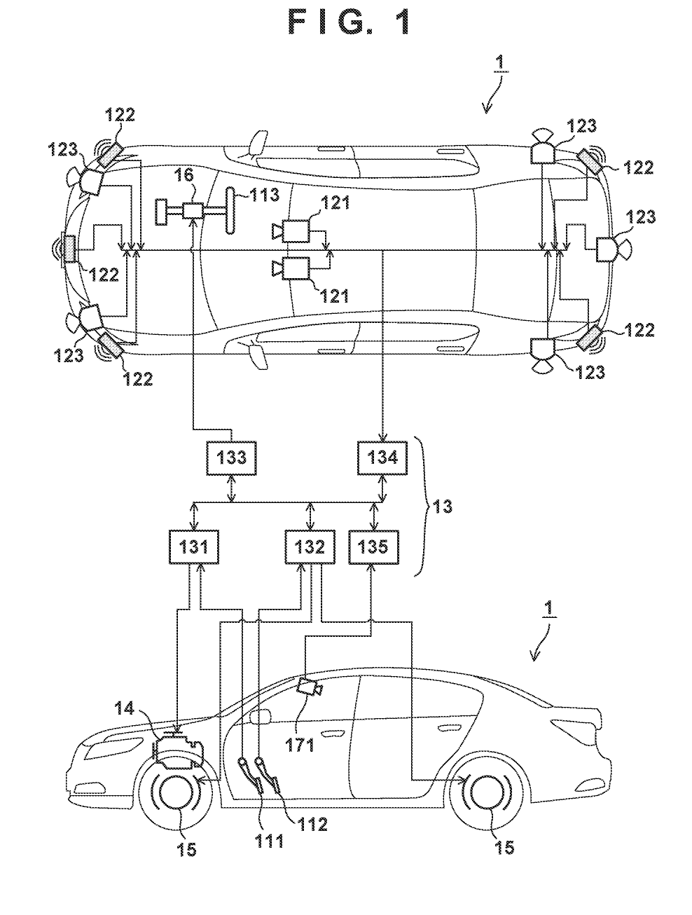

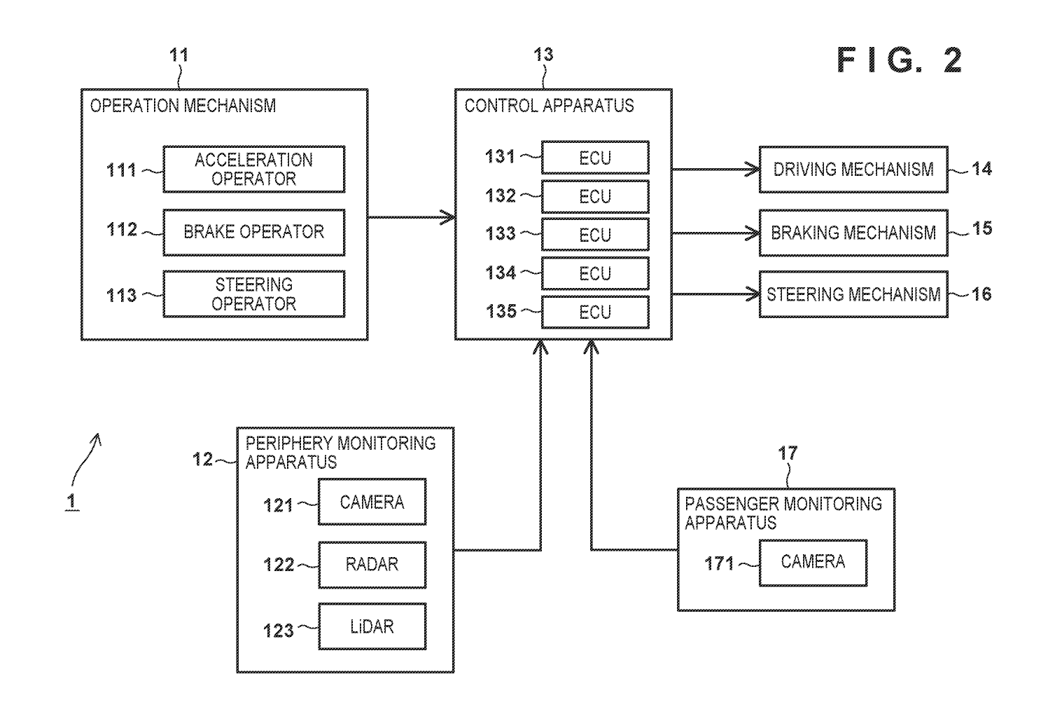

FIGS. 1 and 2 are views for explaining the configuration of a vehicle 1 according to an embodiment. FIG. 1 shows the installation positions of elements to be explained below and the connection relationships between the elements by using a top view and a side view of the vehicle 1. FIG. 2 is a system block diagram of the vehicle 1.

Note that in the following explanation, expressions such as front/rear, upper/lower, and lateral (left/right) will be used in some cases in order to indicate relative directions based on the vehicle body of the vehicle 1. For example, “front” indicates the front in the front-and-rear direction of the vehicle body, and “upper” indicates the direction of height of the vehicle body.

The vehicle 1 includes an operation mechanism 11, a periphery monitoring apparatus 12, a control apparatus 13, a driving mechanism 14, a braking mechanism 15, a steering mechanism 16, and a passenger monitoring apparatus 17. Note that the vehicle 1 is a four-wheeled car in this embodiment, but the number of wheels is not limited to this.

The operation mechanism 11 includes an acceleration operator 111, a brake operator 112, and a steering operator 113. Typically, the acceleration operator 111 is an accelerator pedal, the brake operator 112 is a brake pedal, and the steering operator 113 is a steering wheel. However, each of the operators 111 to 113 may also be another type of an operator such as a lever type operator or a button type operator.

The periphery monitoring apparatus 12 includes cameras 121, radars 122, and LiDARs (Light Detection and Ranging) 123, all of which function as sensors for monitoring or detecting the peripheral environment of the vehicle (self-vehicle) 1. The camera 121 is an imaging device using, for example, a CCD image sensor or a CMOS image sensor. The radar 122 is a distance measurement device such as a millimeter-wave radar. The LiDAR 123 is a distance measurement device such as a laser radar. As exemplarily shown in FIG. 1, these devices are arranged in positions where the peripheral environment of the vehicle 1 can be detected, for example, on the front side, rear side, upper side, and lateral sides of the vehicle body.

Examples of the peripheral environment of the vehicle 1 described above are the traveling environment of the vehicle 1 and the related peripheral environments (for example, the extending direction of a traffic lane, a travelable region, and the color of a traffic signal) of the vehicle 1, and peripheral object information (for example, the presence/absence of an object such as another vehicle, a pedestrian, or an obstacle, and the attribute, the position, and the direction and speed of the movement of the object) of the vehicle 1. From this viewpoint, the periphery monitoring apparatus 12 can also be expressed as a detection device for detecting peripheral information of the vehicle 1.

The control apparatus 13 is capable of controlling the vehicle 1, and controls the mechanisms 14 to 16 based on signals from the operation mechanism 11, the periphery monitoring apparatus 12, and/or the passenger monitoring apparatus 17 (to be described later). The control apparatus 13 includes ECUs (Electronic Control Units) 131 to 135. Each ECU includes a CPU, a memory, and a communication interface. Each ECU causes the CPU to perform predetermined processing based on information (data or an electrical signal) received via the communication interface, stores the processing result in the memory, or outputs the processing result to another element via the communication interface.

The ECU 131 is an acceleration ECU and, for example, controls the driving mechanism 14 (to be described later) based on the amount of operation of the acceleration operator 111 performed by the driver.

The ECU 132 is a braking ECU and, for example, controls the braking mechanism 15 based on the amount of operation of the brake operator 112 performed by the driver. The braking mechanism 15 is, for example, a disk brake formed in each wheel.

The ECU 133 is a steering ECU and, for example, controls the steering mechanism 16 based on the amount of operation of the steering operator 113 performed by the driver. The steering mechanism 16 includes, for example, power steering.

The ECU 134 is an analytical ECU installed for the periphery monitoring apparatus 12. The ECU 134 performs predetermined analysis/processing based on the peripheral environment of the vehicle 1 obtained by the periphery monitoring apparatus 12, and outputs the result to the ECUs 131 to 133. For example, the ECU 134 outputs control signals to the ECUs 131 to 133 so that the vehicle 1 starts/stops in accordance with the color of a traffic signal, and turns along the traffic lane.

The ECU 135 is an analytical ECU installed for the passenger monitoring apparatus 17. In this embodiment, the passenger monitoring apparatus 17 includes a camera 171 installed inside the vehicle, and can obtain an image of a passenger by using the camera 171. As will be described in detail later, the ECU 135 receives this image from the passenger monitoring apparatus 17, evaluates (or, for example, analyzes or estimates) the degree of interest of the passenger with respect to the time, and outputs the result to the ECUs 131 to 133. Like the camera 121, an imaging device such as a CCD/CMOS image sensor can be used as the camera 171.

That is, the ECUs 131 to 133 can control the mechanisms 14 to 16 based on signals from the ECU 134 and/or the ECU 135. With this configuration, the control apparatus 13 can control traveling of the vehicle 1 in accordance with the peripheral environment, for example, can perform automated driving.

In this specification, automated driving is a state in which the control apparatus 13 performs some or all of the driving operations (acceleration, barking, and steering), instead of the driver. That is, the concept of automated driving includes a form (so-called complete automated driving) in which the control apparatus 13 performs all of the driving operations, and a form (so-called drive assist) in which the control apparatus 13 performs only some of the driving operations. Examples of drive assist are a speed control (auto cruise control) function, a distance control (adaptive cruise control) function, a lane departure prevention assist (lane keep assist) function, and a collision avoidance assist function.

Note that the control apparatus 13 is not limited to this configuration. For example, a semiconductor device such as an ASIC (Application Specific Integrated Circuit) may also be used as each of the ECUs 131 to 135. That is, the functions of the ECUs 131 to 135 can be implemented by either hardware or software. Also, some or all of the ECUs 131 to 135 can be configured by a single ECU.

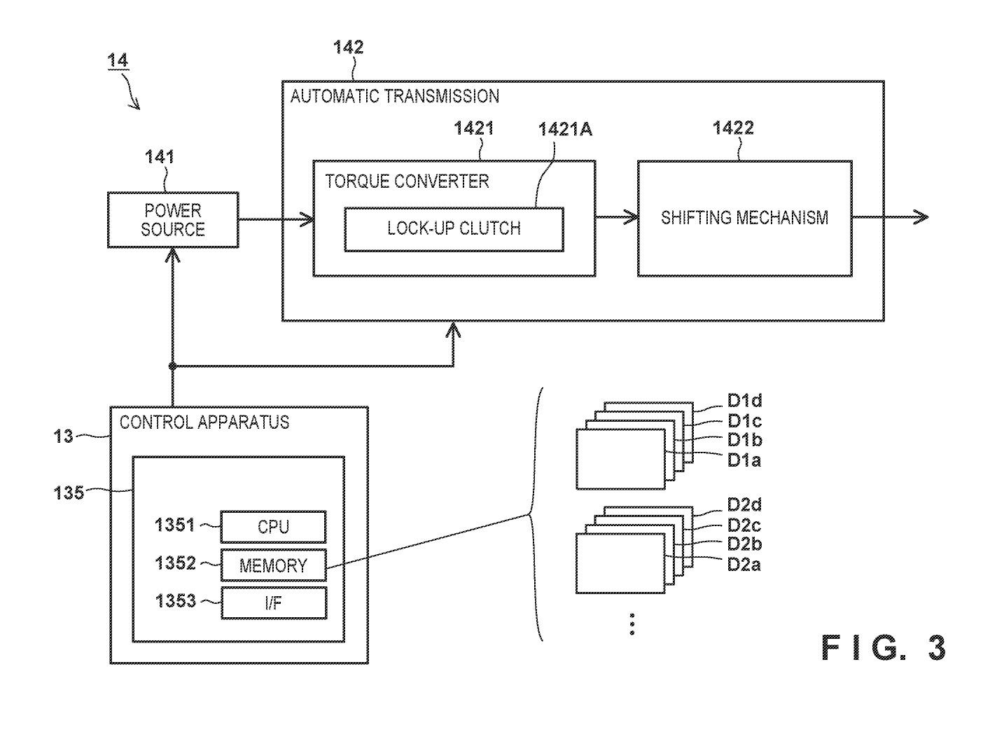

FIG. 3 is a block diagram for explaining portions of the configuration of the vehicle 1, particularly, the driving mechanism 14 and the ECU 135. In this embodiment, the driving mechanism 14 includes a power source 141 and an automatic transmission 142. An internal combustion engine (engine) is used as the power source 141, but it is also possible to use a motor (electric motor) in place of/in addition to the engine as another embodiment. The automatic transmission 142 includes a torque converter 1421 and a shifting mechanism 1422. With this configuration, the automatic transmission 142 shifts the power (engine speed) of the power source 141 based on a predetermined gear ratio, and transmits the shifted power to wheels via a transmitting mechanism (not shown).

The torque converter 1421 is a fluid coupling type starting device arranged between the output shaft of the power source 141 and the input shaft of the shifting mechanism 1422, and can transmit the power of the power source 141 to the shifting mechanism 1422 via a fluid (for example, oil). In this embodiment, a torque converter with a lock-up clutch which includes a lock-up clutch 1421A capable of directly coupling the output shaft of the power source 141 and the input shaft of the shifting mechanism 1422 is used as the torque converter 1421. In a state (directly coupled state) in which the lock-up clutch 1421A is driven and the output shaft of the power source 141 and the input shaft of the shifting mechanism 1422 are mechanically coupled, the power of the power source 141 is directly transmitted to the shifting mechanism 1422. On the other hand, in a state (released state) in which the lock-up clutch 1421A is not driven, the power of the power source 141 is transmitted to the shifting mechanism 1422 via the fluid. When partially driven, the lock-up clutch 1421A can also take an intermediate state (in which the output shaft of the power source 141 and the input shaft of the shifting mechanism 1422 are slidably engaged) between the directly coupled state and the released state. It is also possible to adjust the transmission efficiency of the power of the power source 141 by controlling the driving amount of the lock-up clutch 1421A.

In this embodiment, the shifting mechanism 1422 is a planetary gear type shifting mechanism including a plurality of planetary gear mechanisms and a plurality of engaging mechanisms (for example, clutches and brakes). The shifting mechanism 1422 controls each engaging mechanism based on a signal from the control apparatus 13, and selectively forms one of a plurality of gear ratios by switching transmission paths of the power from the power source 141, thereby determining a gear range. With this configuration, the shifting mechanism 1422 shifts the power of the power source 141 by the gear ratio corresponding to the gear range, and outputs the shifted power.

Assuming that K is an integer of 1 or more, changing a selected gear range from a Kth-speed gear to a (K+1)th-speed gear is called shift-up, and changing a selected gear range from the (K+1)th-speed gear to the Kth-speed gear is called shift-down. An example of shift-up is decreasing the gear ratio by changing the 1st-speed gear to the 2nd-speed gear during acceleration. An example of shift-down is increasing the gear ratio by changing the 4th-speed gear to the 3rd-speed gear during deceleration. Also, shift-up and shift-down are collectively called shift change (a shifting operation).

As described previously, the ECU 135 can receive an image of a passenger from the passenger monitoring apparatus 17, and evaluate the degree of interest of the passenger with respect to the time. In this embodiment, the ECU 135 includes a CPU 1351, a memory 1352, and a communication interface 1353, and performs the abovementioned evaluation by image analysis based on a predetermined program. The memory 1352 holds a look-up table for setting the kinetic characteristic of the vehicle 1. As will be described in detail later, the control apparatus 13 sets the kinetic characteristic of the vehicle 1 by looking up the look-up table corresponding to the result of the evaluation by the ECU 135.

The kinetic characteristic of the vehicle 1 can be changed or adjusted in accordance with the control form (or control mode) of the vehicle 1 during the driving operation. This can be implemented by, for example, changing the setting of the acceleration/deceleration characteristics (the acceleration characteristic and the deceleration characteristic). For example, the kinetic characteristic is improved by raising the acceleration/deceleration characteristics, and restricted by dropping the acceleration/deceleration characteristics. In other words, when the kinetic characteristic improves, an acceleration in the vehicle longitudinal direction to be applied to a passenger during acceleration or deceleration increases. Also, a centrifugal force is applied to the vehicle 1 when it is turning. When the kinetic characteristic improves, therefore, an acceleration in the vehicle left-and-right direction to be applied to a passenger during turning increases. On the other hand, when the kinetic characteristic is restricted, the abovementioned acceleration decreases, and the ride comfort improves.

Note that the expression “the kinetic characteristic, the acceleration/deceleration characteristics, and the like improve” means that the responsiveness of the characteristics improves or a numerical value (for example, a magnitude or intensity) indicating the characteristics increases. For example, improving/raising the kinetic characteristic means that when the driving operation has changed, the behavior of the vehicle 1 corresponding to the change appears within a shorter time. For example, restricting/dropping the acceleration characteristic means that a time before a vehicle speed change corresponding to an accelerating operation (an acceleration instruction by the control apparatus 13 in the case of automated driving) appears prolongs, or an acceleration generated by the accelerating operation decreases.

Since the acceleration/deceleration characteristics follow the control forms of, for example, the power source 141 and the automatic transmission 142, the kinetic characteristic can be changed based on these control forms. For example, when the power source 141 is an internal combustion engine, the power of the power source 141 increases when the fuel injection amount and/or the throttle opening is increased, and this raises the acceleration/deceleration characteristics and improves the kinetic characteristic. For example, in the torque converter 1421 of the automatic transmission 142, when the driving speed of the lock-up clutch 1421A is increased and/or the driving timing thereof is advanced, the power of the power source 141 is transmitted to the shifting mechanism 1422 within a short time, and this raises the acceleration/deceleration characteristics and improves the kinetic characteristic. Also, in the shifting mechanism 1422, when the driving speed of each engaging mechanism such as a clutch or a brake is increased and/or the driving timing thereof is advanced, a time required for shift change (a time required for switching power transmission paths) shortens, and this raises the acceleration/deceleration characteristics and improves the kinetic characteristic.

Note that when the kinetic characteristic improves, the vehicle 1 becomes able to sportily travel, but the acceleration generated in the vehicle 1 and the vibration of the power source 141 generally increase, so the ride comfort worsens. That is, the kinetic characteristic and the ride comfort generally have a trade-off relationship.

In this embodiment as shown in FIG. 3, data D1a to D1d for determining the control form of the power source 141 or the automatic transmission 142 are prepared as a look-up table corresponding to a given kinetic characteristic. For example, the data D1a defines the control setting of the fuel injection amount in the power source 141. For example, the data D1b defines the control setting of the throttle opening in the power source 141. For example, the data D1c defines the control setting such as the driving speed or the driving timing of the lock-up clutch 1421A in the automatic transmission 142. For example, the data D1d defines the control setting such as the driving speed or the driving timing of each engaging mechanism such as a clutch or a brake in the shifting mechanism 1422 of the automatic transmission 142. That is, the abovementioned kinetic characteristic is implemented by controlling the power source 141 and the automatic transmission 142 based on the data D1a to D1d.

Likewise, data D2a to D2d are prepared as a look-up table corresponding to another kinetic characteristic. The data D2a to D2d define the control settings of the other kinetic characteristic in one-to-one correspondence with the contents of the data D1a to D1d, and the other kinetic characteristic is implemented by controlling the power source 141 and the automatic transmission 142 based on the data D2a to D2d. Although not shown in FIG. 3, this also applies to a look-up table corresponding to still another kinetic characteristic.

Note that several look-up tables may also be combined as another embodiment. For example, it is possible to selectively implement various kinetic characteristic by controlling the power source 141 based on the data D1a and D1b, and controlling the automatic transmission 142 based on the data D2c and D2d.

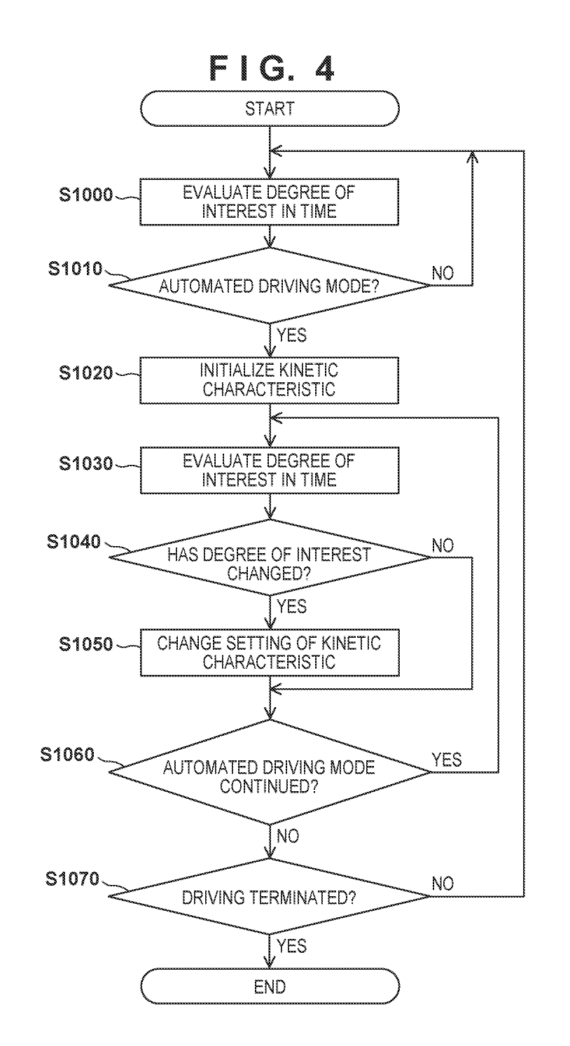

FIG. 4 is a flowchart showing a method of setting the kinetic characteristic of the vehicle 1 according to this embodiment. The control apparatus 13 (mainly the ECU 135) performs the contents of this setting method. This flowchart is performed in accordance with driving start. Note that “driving start” is to set the vehicle 1 in a travelable state, for example, in an ignition ON state. An outline of the method is to start automated driving based on a predetermined kinetic characteristic, and continue automated driving while changing the setting of the kinetic characteristic in accordance with the degree of interest of a passenger with respect to the time.

First, in step S1000 (to be simply referred to as “S1000” hereinafter; this also applies to other steps), the degree of interest of a passenger with respect to the time is evaluated. The degree of interest is an index indicating an extent to which the passenger cares the time, and is generally evaluable based on the frequency at which the passenger looks at a clock. As will be described in detail later, the frequency of looking can be determined by, for example, the number of times the passenger looks at a clock within a predetermined time (for example, 10 minutes, 30 minutes, or 1 hour). When the frequency of looking is relatively high, it can be estimated that the passenger cares the time for some reason, for example, he or she is in a hurry to arrive at a predetermined destination. Note that the abovementioned degree of interest will simply be expressed as “the degree of interest” in some cases in the following explanation.

The degree of interest can be evaluated by analyzing an image of a passenger obtained by the camera 171 of the passenger monitoring apparatus 17. This evaluation generates an evaluation value indicating the degree of interest.

Examples of an evaluation tool for evaluating the degree of interest are Sentiment Analysis Glassware (Emotient) and Emospark (EmoShape). It is also possible to use, for example, FMH (Flicker Health Management), JINS MEME (JIN), or Monitoring System (CAARESYS) in place of/in addition to the camera 171.

In this embodiment, a passenger in the driver's seat (that is, a driver) is a target of the evaluation of the degree of interest in order to simplify the explanation. As another embodiment, however, a passenger in the passenger's seat or in the rear seat can also be a target of the evaluation of the degree of interest. Furthermore, S1000 may also be omitted if automated driving is started immediately after driving start.

Then, in S1010, whether the operation mode of the vehicle 1 changes to an automated driving mode is determined. If the operation mode changes to the automated driving mode, the process advances to S1020. If the operation mode does not change to the automated driving mode (that is, if a normal mode continues), the process returns to S1000. That is, in this embodiment, the evaluation of the degree of interest is repeatedly performed even when automated driving is not started.

Note that switching of the operation modes from one of the automated driving mode and the normal mode to the other can be performed when the user presses a predetermined switch in the vehicle. The user herein mentioned is a person who can be a driver when automated driving is canceled.

In S1020, in response to the determination in S1010 that automated driving is to be started, initial setting of the kinetic characteristic of the vehicle 1 according to the automated driving is performed. In this embodiment, setting based on the evaluation result (the evaluation value of the degree of interest) in S1000 is selected as the kinetic characteristic. For example, if the degree of interest of the passenger with respect to the time is relatively low, a control form based on the data D1a to D1d (see FIG. 3) is determined as the control form of the power source 141 and the automatic transmission 142. Also, if this degree of interest is relatively high, a control form based on the data D2a to D2d (see FIG. 3) is determined as the control form of the power source 141 and the automatic transmission 142.

Note that as another embodiment, it is possible to select, in S1020, predetermined setting or setting customized by the user in advance, as the kinetic characteristic at the start of automated driving. In this case, S1000 described above may also be omitted.

In S1030, the degree of interest of the passenger of the vehicle 1 with respect to the time is evaluated. S1030 need only be performed following the same procedure as in S1000 described above. This evaluation in S1030 updates the evaluation value of the degree of interest from that in S1000.

In S1040, whether the degree of interest has changed, that is, whether the evaluation value of the degree of interest has a predetermined change or more is determined. If the evaluation value of the degree of interest has the predetermined change or more, the process advances to S1050; if not, the process advances to S1060.

In S1050, the setting of the kinetic characteristic is changed. For example, if the evaluation value of the degree of interest becomes higher than a reference range (or a reference value), it is estimated that the passenger is relatively in a hurry, so the kinetic characteristic is improved. That is, this kinetic characteristic is changed from the kinetic characteristic set in S1020 to a sportier kinetic characteristic. If the evaluation value of the degree of interest becomes lower than the reference range, it is estimated that the passenger is not particularly in a hurry (or that the vehicle 1 can arrive at a time planned by the occupant with time to spare), so the kinetic characteristic is restricted. That is, this kinetic characteristic is changed from the kinetic characteristic set in S1020 to a milder kinetic characteristic.

In S1060, whether to continue the automated driving mode as the operation mode of the vehicle 1 is determined. If the automated driving mode is to be continued, the process returns to S1030; if not, the process advances to S1070. That is, in S1030 to S1060, while automated driving is performed, the degree of interest is evaluated based on an image obtained by the camera 171, and the kinetic characteristic of the vehicle 1 is changed to a kinetic characteristic corresponding to the evaluation result.

In S1070, whether to terminate driving is determined. If driving is to be terminated, this process is terminated; if not, the process returns to S1000. Note that driving termination is to set the vehicle 1 in an untravelable state, for example, in an ignition OFF state.

As described above, traveling control appropriate for a passenger can be implemented by performing automated driving while changing the setting of the kinetic characteristic of the vehicle 1 to a kinetic characteristic corresponding to the degree of interest of the passenger with respect to the time. For example, when it is estimated that the passenger is in a hurry, relatively sporty traveling control is performed by a high kinetic characteristic. On the other hand, when it is estimated that the passenger is not particularly in a hurry, a comfortable ride can be provided to the passenger by performing relatively mild traveling control by a restricted kinetic characteristic.

The degree of interest can be evaluated relatively easily by determining whether the passenger looks at a clock installed in the vehicle. As another example, this evaluation can also be performed by another method such as a method of determining whether the passenger raises his or her arm and checks a wristwatch. In this embodiment, whether the passenger looks at a clock (“a clock 18”) in the vehicle is determined. In this case, the camera 171 is preferably arranged in a position where it is possible to detect whether the passenger looks at the clock 18. For example, the camera 171 is preferably arranged near the clock 18. As an example, the camera 171 can be integrated with the clock 18. This makes it possible to more appropriately determine whether the passenger looks at the clock 18.

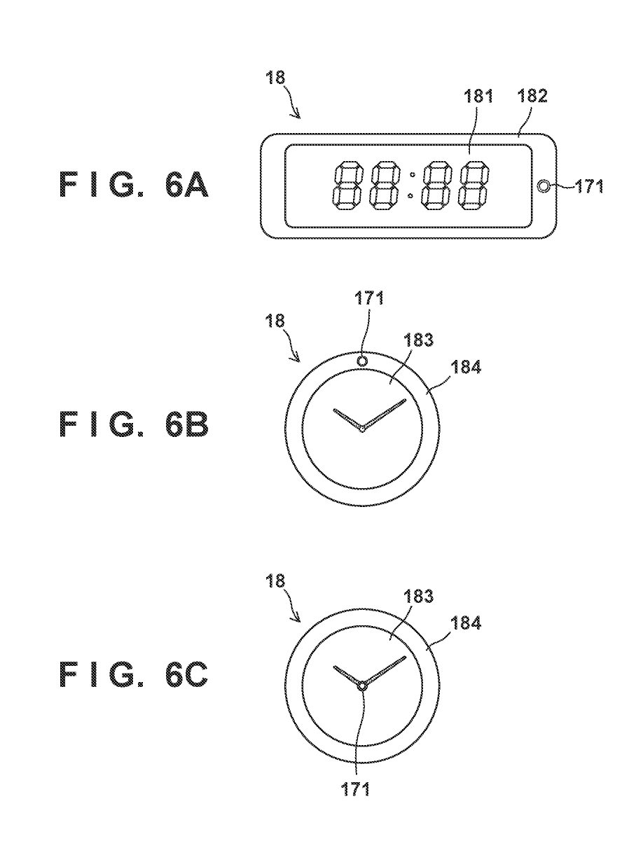

As shown in FIG. 6A, an example of the clock 18 is a digital clock. In this example shown in FIG. 6A, the clock 18 includes a liquid crystal display unit 181 for displaying the time and a frame 182 surrounding the display unit 181, and the camera 171 can be formed in the frame 182. The camera 171 can also be formed in the frame 182 as a part of the display unit 181.

As shown in FIGS. 6B and 6C, other examples of the clock 18 are analog clocks. In these examples shown in FIGS. 6B and 6C, the clock 18 includes a clock face 183 and a frame 184 surrounding the clock face 183. The camera 171 can be formed in the frame 184 as shown in FIG. 6B, and can also be formed in the clock face 183 as shown in FIG. 6C. Note that in the example shown in FIG. 6C, the camera 171 is formed in the shafts of the long hand and the short hand, so the camera 171 can image the interior of the vehicle without being hidden behind the long hand and the short hand.

In the examples shown in FIGS. 6A to 6C, the camera 171 can appropriately detect that the passenger looks at the clock 18. As the camera 171, a compact camera such as a CCD/CMOS image sensor can be used.

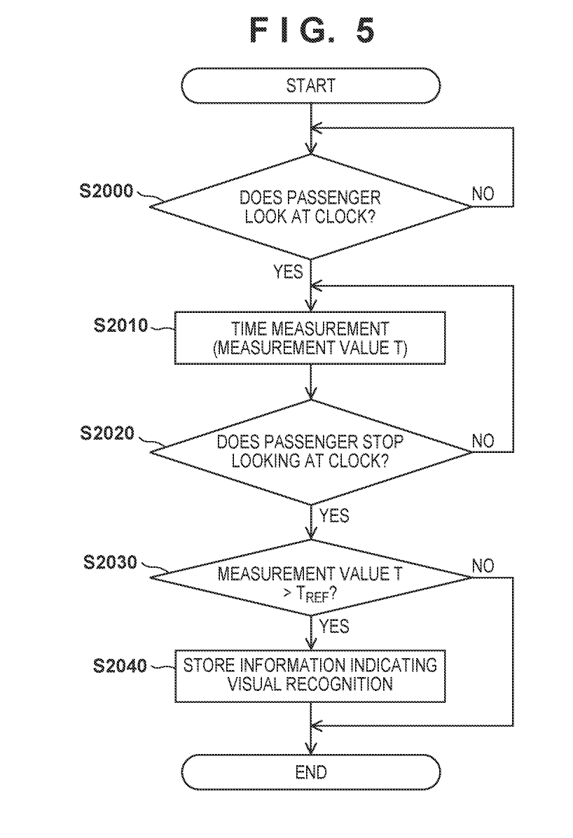

FIG. 5 is a flowchart showing a part of the method of evaluating the degree of interest in S1000 and S1030 (see FIG. 4) described above. The control apparatus 13 (mainly the ECU 135) performs the contents of this evaluation method. The evaluation of the degree of interest according to this embodiment is continuously performed after the start of driving and before the end of driving as described above with reference to FIG. 4. An outline of the evaluation is to determine whether the passenger looks at the clock 18 based on an image obtained by the camera 171, and measure the number of times of looking.

In S2000, whether the passenger looks at the clock 18 is determined. S2000 is performed based on an image obtained by the camera 171. If the passenger looks at the clock 18, the process advances to S2010; if not, the process returns to S2000.

In S2010, a time during which the passenger is looking at the clock 18 is measured. S2010 is performed by using, for example, a count-up timer, and the time having elapsed since the passenger starts looking at the clock 18 is obtained as a measurement time T.

In S2020, whether the passenger stops looking at the clock 18 is determined. S2020 is performed based on an image obtained by the camera 171. If the passenger stops looking at the clock 18, the process advances to S2030; if not, the process returns to S2010. That is, a period during which the passenger keeps looking at the clock 18 is obtained as the measurement value T in S2010 and S2020.

In S2030, whether the measurement value T obtained in S2010 and S2020 is larger than a predetermined value T_{REF} is determined. S2030 can be performed by using a comparator which compares the values of T and T_{REF}. If T>T_{REF}, it is determined that the passenger looks at the clock 18, and the process advances to S2040; if not, this process is terminated. Note that T_{REF} can be fixed to a predetermined time (for example, 0.1 to 2.0 sec) generally required for a person to recognize the time by looking at the clock 18, and can also be set by the user.

In S2040, since it is determined in S2030 that the passenger looks at the clock 18, information indicating that the passenger looks at the clock 18 (one-time looking) is temporarily stored in the memory 1352 by the CPU 1351 or the like.

By referring to the memory 1352, the ECU 135 can measure the number of times the passenger looks at the clock 18 during a predetermined period (for example, 10 minutes, 30 minutes, or 1 hour) before the present time. The ECU 135 generates the measurement value of the number of times of looking obtained as described above, as an evaluation value of the degree of interest of the passenger with respect to the time. The ECU 135 can perform the evaluation of the degree of interest (that is, the generation of the evaluation value of the degree of interest) as described above at a predetermined period.

Note that if T T_{REF} in S2030, it is determined that the passenger does not look at the clock 18. Therefore, if the eyes of the passenger accidentally pass the clock 18 but the passenger does not look at the clock 18, this eye movement is not unnecessarily counted as the number of times of looking. This makes it possible to appropriately evaluate the degree of interest.

The above-described degree-of-interest evaluation results are preferably accumulated in, for example, the memory 1352. The CPU 1351 can refer to the accumulated evaluation results as the past evaluation results. In S1040, therefore, a change in degree of interest can properly be determined. That is, a change in degree of interest can be monitored by comparing the frequency at which the passenger looks at the clock 18 within a predetermined period, with the frequency of looking in the past before the predetermined period. Also, the degree of interest can have individual differences. In this embodiment, therefore, it is also possible to monitor a change in degree of interest by taking the individual differences into consideration.

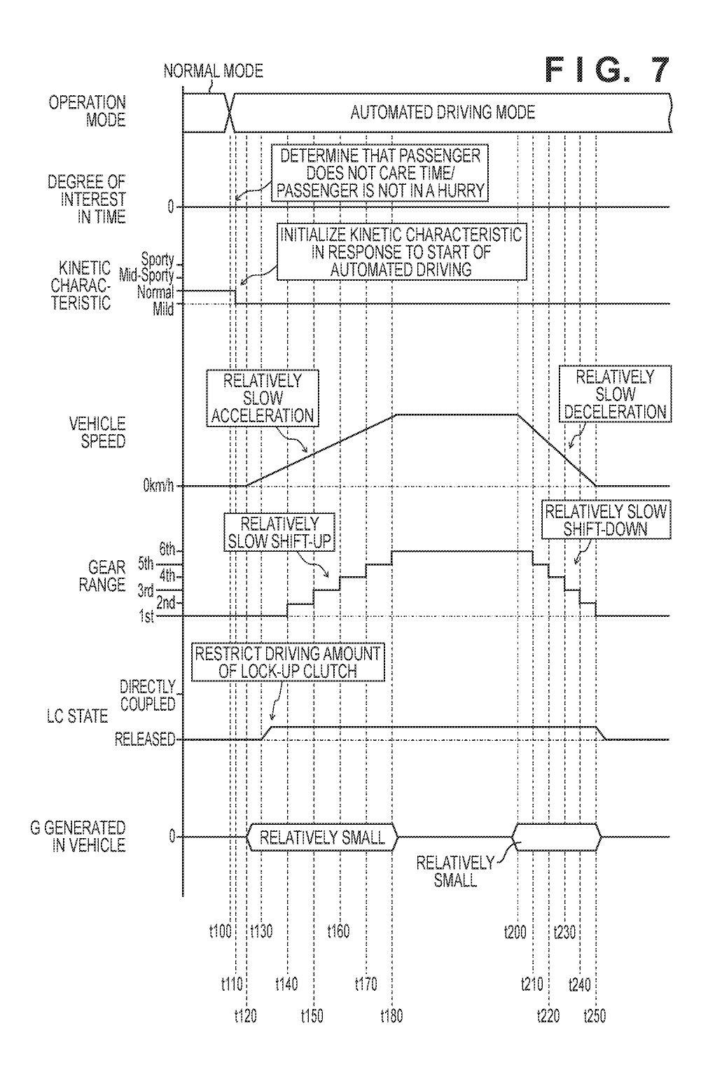

FIG. 7 is a timing chart showing the first example of the setting form of the kinetic characteristic of the vehicle 1. In FIG. 7, the abscissa indicates the time axis, and the ordinate indicates “operation mode” of the vehicle 1, “degree of interest in time” of the passenger, “kinetic characteristic” and “vehicle speed” of the vehicle 1, “gear range” of the shifting mechanism 1422, “LC state” as the state of the lock-up clutch 1421A, and “G (acceleration) generated in vehicle”.

The operation mode includes the automated driving mode in which the control apparatus 13 performs at least some of the driving operations, and the normal mode in which the driver performs all of the driving operations. As described previously, the user can switch the operation modes by pressing a predetermined switch in the vehicle.

As described earlier, the degree of interest in time shows an extent to which the passenger cares the time. For example, as the degree of interest becomes higher, the passenger hopes to/is in a hurry to arrive at a destination earlier.

The kinetic characteristic shows which of four kinetic characteristic, that is, a standard kinetic characteristic (Normal), a restricted kinetic characteristic (Mild), an improved kinetic characteristic (Sporty), and a slightly improved kinetic characteristic (Mid-Sporty), is set. When these kinetic characteristic are rearranged in ascending order from the lowest one, the result is Mild, Normal, Mid-Sporty, and Sporty.

The gear range shows six ranges, that is, the 1st-speed gear to the 6th-speed gear in this example, but the number of gear ranges is not limited to this. Note that parking (P), reverse (R), and neutral (N) are also provided although they are not shown because they are not used herein.

The LC state includes the directly coupled state in which the lock-up clutch 1421A is driven, the released state in which the lock-up clutch 1421A is not driven, and the intermediate state between them (see FIG. 3). The power transmission efficiency of the power source 141 can be adjusted by controlling the LC state. For example, the power transmission efficiency of the power source 141 is maximum in the directly coupled state, and partially restricted in the intermediate state.

To simplify the understanding, G generated in vehicle indicates G caused by the acceleration generated when, for example, the vehicle starts, or by the vibration of the power source 141, and does not include G caused by another external element (for example, a vibration generated by the slope or unevenness of the road surface itself). For example, when acceleration is performed with a relatively high kinetic characteristic, a relatively large G is generated. However, G which can be generated when the vehicle is turning or climbing a slope is not shown.

First, at time t100, the operation mode of the vehicle 1 changes from the normal mode to the automated driving mode (see S1010). In the first example, the degree of interest of the passenger with respect to the time is low/practically zero (see S1000), so the kinetic characteristic is changed to the restricted setting (Mild) at time t110 (see S1020). At time t120, the output (speed) of the power source 141 is increased and transmitted to the wheels via the automatic transmission 142, so the vehicle 1 starts and raises the speed. At time t130, as the vehicle speed rises, the LC state is changed to the above-described intermediate state by partially driving the lock-up clutch 1421A. At times t140 to t180, the gear ranges are shifted up in order, that is, the 2nd-speed gear at time t140, the 3rd-speed gear at time t150, the 4th-speed gear at time t160, the 5th-speed gear at time t170, and the 6th-speed gear at time t180.

In the first example, the kinetic characteristic is fixed to the restricted setting (Mild) because the degree of interest of the passenger with respect to the time is low. Accordingly, after time t120 at which the output of the power source 141 is increased to start the vehicle 1, G caused by the acceleration or the vibration of the power source 141 is relatively small in a period during which the vehicle 1 starts and reaches a predetermined speed (from the start to the completion of acceleration).

The abovementioned G is the same at times t200 to t250 from the start to the stop of deceleration. At time t200, the vehicle 1 starts decelerating. At times t210 to t250 after that, the gear ranges are shifted down in order, that is, the 5th-speed gear at time t210, the 4th-speed gear at time t220, the 3rd-speed gear at time t230, the 2nd-speed gear at time 240, and the 1st-speed gear at time t250. In addition, the LC state is changed to the released state at time t250. In the first example, since the kinetic characteristic is fixed to the restricted setting (Mild), G caused by the acceleration or the vibration of the power source 141 is relatively small from the start to the completion of deceleration.

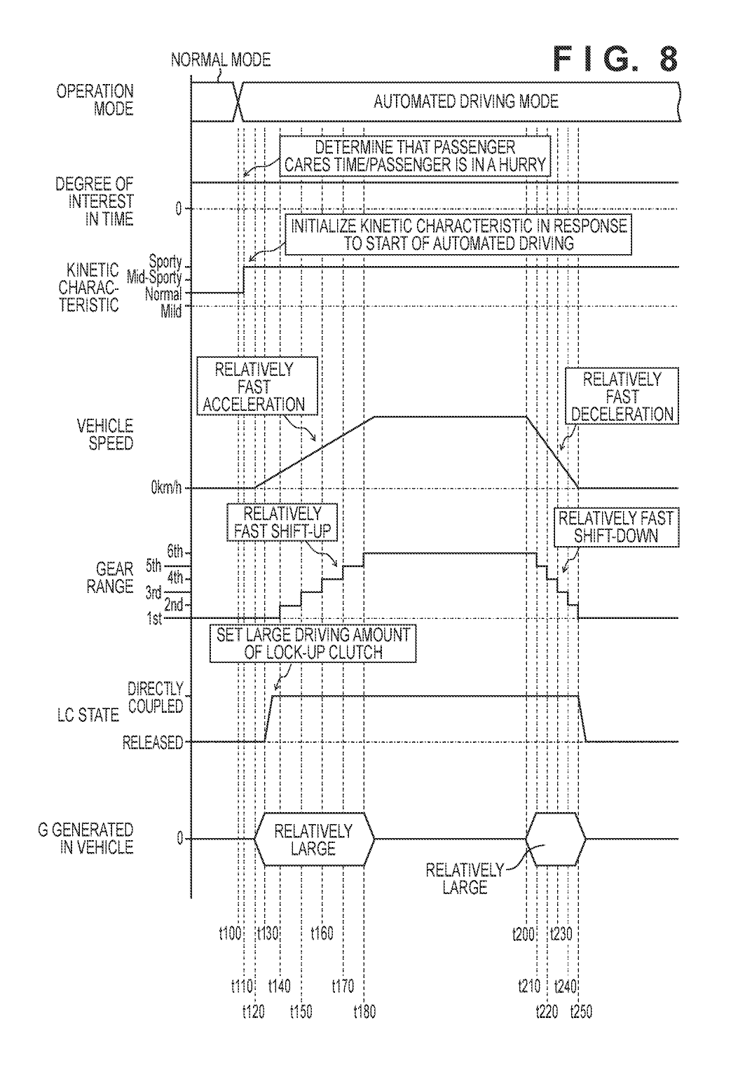

FIG. 8 is a timing chart showing the second example of the setting form of the kinetic characteristic. The second example differs from the above-described first example in that the degree of interest of the passenger with respect to the time is high when automated driving is started.

In the second example, automated driving is started and the kinetic characteristic is initially set at times t100 and t110 (as in the first example). After that, if it is detected that the degree of interest is high (see S1000), the setting of the kinetic characteristic is changed (see S1020). In the second example, the kinetic characteristic is changed to the setting of the abovementioned improved kinetic characteristic (Sporty).

In the second example, the behaviors at times t120 to t180 are the same as those in the first example, but the setting of the kinetic characteristic is fixed to the improved one (Sporty). Therefore, the intervals of times t120 to t180 in the second example are shorter than those in the first example. Accordingly, after time t120 at which the output of the power source 141 is increased, G generated by the acceleration or the vibration of the power source 141 is larger than that in the first example from the start to the completion of acceleration. For example, at time t130, the driving force of the lock-up clutch 1421A is increased by changing the LC state to the directly coupled state. In the second example, therefore, the abovementioned G is larger than that in the first example.

This similarly applies to the abovementioned G at times t200 to t250 from the start to the stop of deceleration. In the second example, the behaviors at times t200 to t250 are the same as those in the first example, but the kinetic characteristic is set to the improved kinetic characteristic (Sporty). This increases G generated from the start to the completion of deceleration.

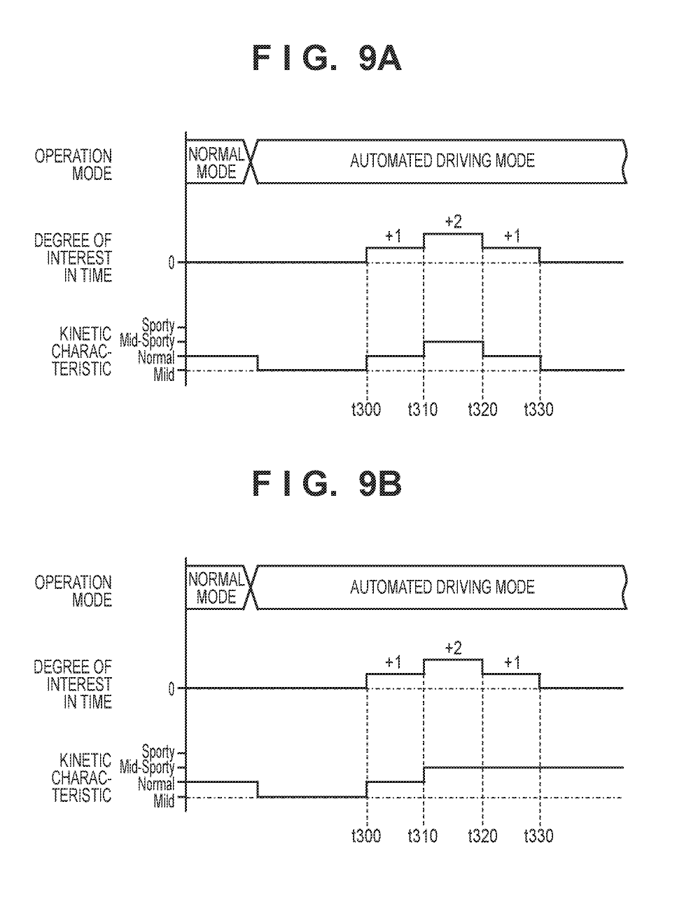

As the third example, FIG. 9A shows the setting form of the kinetic characteristic when the degree of interest of the passenger with respect to the time has changed after the operation mode of the vehicle 1 is changed to the automated driving mode. In the third example, the degree of interest is practically zero (degree of interest±0) when automated driving is started, so the kinetic characteristic is set to the restricted one (Mild). At time t300 after that, the degree of interest rises by one step (degree of interest+1), and the setting of the kinetic characteristic is changed to the standard one (Normal) accordingly. At time t310, the degree of interest further rises by one step (degree of interest+2), and the setting of the kinetic characteristic is changed to the slightly improved one (Mild-Sporty) accordingly. At time t320 after that, the degree of interest drops by one step (degree of interest+1), and the setting of the kinetic characteristic is changed to the standard one (Normal) again accordingly. Furthermore, at time t330 after that, the degree of interest further drops by one step (degree of interest±0), and the setting of the kinetic characteristic is changed to the restricted one (Mild) again accordingly.

That is, in the third example, after automated driving is started, the setting of the kinetic characteristic is changed in accordance with the change in degree of interest of the passenger with respect to the time (see S1030 to S1050). Note that the forms of acceleration/deceleration corresponding to the individual settings of the kinetic characteristic are the same as those in the first and second examples (see FIGS. 7 and 8). As described above, traveling control appropriate for the passenger can be implemented by performing automated driving while changing the kinetic characteristic of the vehicle 1 to the setting corresponding to the degree of interest of the passenger with respect to the time. For example, when it is estimated that the passenger is in a hurry, relatively sporty traveling control is performed by a high kinetic characteristic. On the other hand, when it is estimated that the passenger is not particularly in a hurry, a comfortable ride can be provided to the passenger by performing relatively mild traveling control by a restricted kinetic characteristic.

As the fourth example, FIG. 9B shows another setting form of the kinetic characteristic when the degree of interest of the passenger with respect to the time changes after the operation mode of the vehicle 1 is changed to the automated driving mode. The fourth example differs from the third example in that the kinetic characteristic is improved when the degree of interest rises and the improved kinetic characteristic is maintained when the degree of interest drops. That is, when it is assumed that the passenger can arrive at a scheduled time with plenty of time remaining, the kinetic characteristic is not restricted. Note that it is favorable to allow the user to preselect the third or fourth example as the kinetic characteristic setting form corresponding to the degree of interest.

Alternatively, as a modification of the fourth example, it is also possible to decrease the kinetic characteristic if a predetermined time (for example, 10 minutes, 30 minutes, or 1 hour) has elapsed. In this case, the passenger can arrive at a scheduled time with time to spare, and the vehicle 1 is traveling relatively mildly at the time of arrival. As a consequence, the load on the passenger can be reduced by providing a comfortable ride.

As still another example, a predetermined hysteresis characteristic can be given to the correlation between the setting of the kinetic characteristic and the degree of interest. For example, after the degree of interest changes from ±0 to +1 and the setting of the kinetic characteristic is changed to a higher one, the changed setting is maintained when the degree of interest returns to ±0 again. On the other hand, after the degree of interest changes from ±0 to +2 and the setting of the kinetic characteristic is changed to a higher one, the changed setting is returned to the original one when the degree of interest returns to ±0 again.

In this embodiment as described above, the degree of interest of the passenger of the vehicle 1 with respect to the time is evaluated, and the setting of the kinetic characteristic of the vehicle 1 is changed based on the evaluation result. Since the kinetic characteristic of the vehicle 1 matches the degree of interest of the passenger with respect to the time, traveling control appropriate for the passenger can be implemented.

In this embodiment, the degree of interest of the passenger with respect to the time is evaluated even in the normal mode (S1000). However, this evaluation may also be omitted in the normal mode as described previously. In this case, the initial setting of the kinetic characteristic when the automated driving mode is set can be the standard one (Normal).

Also, in this embodiment, the degree of interest of the passenger in the driver's seat is the evaluation target. However, a passenger not in the driver's seat (for example, a passenger in the passenger's seat or in the rear seat) can also be a target of the evaluation of the degree of interest. In this case, it is possible to evaluate the degree of interest of each of a plurality of passengers, and add the evaluation values by weighted addition. Note that coefficients to be used in this weighted addition can be set such that a coefficient corresponding to a passenger in the driver's seat is larger than coefficients corresponding to other passengers. It is also possible to use equal values for all passengers. Traveling control appropriate for a plurality of passengers can be implemented by changing the setting of the kinetic characteristic of the vehicle 1 based on the result of the above-described weighted addition.

The present invention is not limited to the above-described examples, and various modifications are applicable. For example, the configuration of the driving mechanism 14 is not limited to the example shown in FIG. 3, and another well-known configuration may also be adopted. For example, a dual clutch transmission (DCT) can be used as the automatic transmission 142.

Furthermore, in the above-described examples, the kinetic characteristic of the vehicle 1 is improved or restricted by changing the control forms of the power source 141 and the automatic transmission 142. However, another method may also be adopted. For example, the kinetic characteristic also follows the damping characteristic of a suspension mechanism (not shown) for absorbing vibrations from the road surface. Therefore, if the vehicle 1 includes a suspension mechanism having an adjustable damping characteristic, the setting of the kinetic characteristic of the vehicle 1 can be changed by adjusting this damping characteristic.

In the embodiment, a form in which the kinetic characteristic is set to the standard one (Normal) in the normal mode and the setting of the kinetic characteristic is changed in the automated driving mode has been exemplified. However, this is also applicable to the normal mode. That is, even in the normal mode, the kinetic characteristic of the vehicle 1 can be changed to the setting corresponding to the degree of interest of the passenger with respect to the time.

In addition, the individual terms described in this specification are merely used to explain the present invention, and the present invention is, of course, not limited to the strict meanings of these terms and can include their equivalents.

The features of the present invention will be summarized below:

The first aspect is a control apparatus (for example, 13, 135) for controlling a vehicle (for example, 1), comprising an evaluation unit (for example, 135, S1020) configured to evaluate a degree of interest of a passenger of the vehicle with respect to time, and a setting changing unit (for example, 135, S1040) configured to change setting of a kinetic characteristic of the vehicle based on an evaluation result obtained by the evaluation unit.

According to the first aspect, traveling control appropriate for the passenger can be implemented by matching the kinetic characteristic of the vehicle with the degree of interest of the passenger with respect to the time.

In the second aspect, the vehicle includes an automated driving mode as an operation mode (for example, S1000, S1050), and, in the automated driving mode, the evaluation unit evaluates the degree of interest, and the setting changing unit changes the setting.

According to the second aspect, the abovementioned traveling control can suitably be implemented in the automated driving mode (including a driving assist mode).

In the third aspect, the setting changing unit changes the setting of the kinetic characteristic by changing setting of acceleration/deceleration characteristics of the vehicle.

According to the third aspect, the setting of the acceleration/deceleration characteristics can be changed by changing, for example, a control form of a driving mechanism. This makes it possible to relatively simply set the kinetic characteristic of the vehicle.

In the fourth aspect, the vehicle includes an automatic transmission (for example, 142), and the setting changing unit changes the setting of the acceleration/deceleration characteristics by changing a control form of the automatic transmission.

According to the fourth aspect, the setting of the acceleration/deceleration characteristics can be changed by changing the control form of the automatic transmission as a part of the driving mechanism. Therefore, it is possible to relatively simply change the setting of the kinetic characteristic of the vehicle.

In the fifth aspect, the automatic transmission includes a shifting mechanism (for example, 1422), and the setting changing unit changes the setting of the acceleration/deceleration characteristics by changing a control form of an engaging mechanism of the shifting mechanism.

According to the fifth aspect, the setting of the acceleration/deceleration characteristics can be changed by changing the control form of the shifting mechanism. This makes it possible to relatively simply change the setting of the kinetic characteristic of the vehicle. When using a planetary gear type shifting mechanism, for example, an example of the control form is to change the driving speed and/or the driving timing of the engaging mechanism such as a clutch or a brake.

In the sixth aspect, the automatic transmission includes a torque converter (for example, 1421) with a lock-up clutch (for example, 1421A), and the setting changing unit changes the setting of the acceleration/deceleration characteristics by changing a control form of the lock-up clutch.

According to the sixth aspect, the setting of the acceleration/deceleration characteristics can be changed by changing the control form of the lock-up clutch. Therefore, it is possible to relatively simply change the setting of the kinetic characteristic of the vehicle. An example of the control form of the lock-up clutch is to change the driving speed and/or the driving timing of the lock-up clutch.

In the seventh aspect, the setting changing unit improves the kinetic characteristic when the degree of interest evaluated by the evaluation unit is higher than a reference range, and restricts the kinetic characteristic when the degree of interest is lower than the reference range.

According to the seventh aspect, when it is estimated that the passenger does not care the time (the passenger is not in a hurry), a comfortable ride is provided to the passenger by performing traveling control which restricts the kinetic characteristic of the vehicle. On the other hand, when it is estimated that the passenger cares the time (the passenger is in a hurry), stress-free traveling control is performed by performing traveling control with a high vehicle kinetic characteristic. This can implement traveling control appropriate for the passenger.

In the eighth aspect, the vehicle includes an imaging apparatus (for example, 171) installed in the vehicle, and the evaluation unit evaluates the degree of interest based on an image obtained by the imaging apparatus.

According to the eighth aspect, it is possible to appropriately monitor the state of a passenger in the vehicle, and appropriately evaluate the degree of interest of the passenger with respect to the time.

In the ninth aspect, the vehicle includes a clock (for example, 18) installed in the vehicle, and the evaluation unit evaluates the degree of interest based on a frequency at which the passenger looks at the clock.

According to the ninth aspect, it is possible to appropriately evaluate the degree of interest of the passenger with respect to the time. The frequency of looking can be determined by the number of times of looking within a predetermined time.

In the 10th aspect, the vehicle includes a clock (for example, 18) installed in the vehicle, and an imaging apparatus (for example, 171) integrated with the clock.

According to the 10th aspect, it is possible to appropriately detect that the passenger looks at the clock.

The 11th aspect further includes a comparison unit configured to compare the frequency at which the passenger looks at the clock within a predetermined period with the frequency at which the passenger looks at the clock before the predetermined period, and the evaluation unit evaluates the degree of interest based on the result of comparison by the comparison unit.

According to the 11th aspect, the setting of the kinetic characteristic can be changed in accordance with, for example, a change in degree of interest, by referring to the frequency of looking in the past (the evaluation result of the degree of interest in the past). This is advantageous in performing traveling control taking account of individual differences of the degree of interest.

The 12th aspect includes a measurement unit configured to, when the passenger looks at the clock, measure a time during which the passenger is looking at the clock, and a determination unit configured to determine that the passenger looks at the clock if the measurement value obtained by the measurement unit is larger than a predetermined value.

According to the 12th aspect, the number of times the passenger looks at the clock is not unnecessarily measured. For example, if the eyes of the passenger accidentally pass the clock, this eye movement is not counted as the number of times of looking. This makes it possible to more appropriately change the setting of the kinetic characteristic.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A control apparatus for controlling a vehicle, comprising:
an evaluation unit configured to evaluate a degree of interest of a passenger of the vehicle with respect to time; and
a setting changing unit configured to change setting of a kinetic characteristic of the vehicle based on an evaluation result obtained by the evaluation unit.

2. The apparatus according to claim 1, wherein
the vehicle includes an automated driving mode as an operation mode, and
in the automated driving mode, the evaluation unit evaluates the degree of interest, and the setting changing unit changes the setting.

3. The apparatus according to claim 1, wherein the setting changing unit changes the setting of the kinetic characteristic by changing setting of acceleration/deceleration characteristics of the vehicle.

4. The apparatus according to claim 3, wherein
the vehicle includes an automatic transmission, and
the setting changing unit changes the setting of the acceleration/deceleration characteristic by changing a control form of the automatic transmission.

5. The apparatus according to claim 4, wherein
the automatic transmission includes a shifting mechanism, and
the setting changing unit changes the setting of the acceleration/deceleration characteristic by changing a control form of an engaging mechanism of the shifting mechanism.

6. The apparatus according to claim 4, wherein
the automatic transmission includes a torque converter with a lock-up clutch, and
the setting changing unit changes the setting of the acceleration/deceleration characteristic by changing a control form of the lock-up clutch.

7. The apparatus according to claim 1, wherein the setting changing unit improves the kinetic characteristic when the degree of interest evaluated by the evaluation unit is higher than a reference range, and restricts the kinetic characteristic when the degree of interest is lower than the reference range.

8. The apparatus according to claim 1, wherein
the vehicle includes an imaging apparatus installed in the vehicle, and
the evaluation unit evaluates the degree of interest based on an image obtained by the imaging apparatus.

9. The apparatus according to claim 1, wherein
the vehicle includes a clock installed in the vehicle, and
the evaluation unit evaluates the degree of interest based on a frequency at which the passenger looks at the clock.
